(19) United States
(12) Patent Application Publication
(10) Pub. No.: US 2019/0117655 A1
(43) Pub. Date: Apr. 25, 2019

(54) Treatment of Biliary Duct Cancer

(71) Applicant: ASLAN Pharmaceuticals PTE LTD, Singapore (SG)

(72) Inventors: Bertil Lindmark, Singapore (SG); Mark Thomas McHale, Singapore (SG); Lisa Ooi, Singapore (SG)

(73) Assignee: ASLAN Pharmaceuticals PTE LTD, Singapore (SG)

(21) Appl. No.: 16/134,736

(22) Filed: Sep. 18, 2018

Related U.S. Application Data
(63) Continuation-in-part of application No. 15/757,166, filed on Mar. 2, 2018.
(60) Provisional application No. 62/217,332, filed on Sep. 11, 2015.
(60) Provisional application No. 62/217,346, filed on Sep. 11, 2015.

(30) Foreign Application Priority Data
Sep. 4, 2015 (GB) 1515712.6
Sep. 4, 2015 (GB) 1515714.2
Sep. 4, 2015 (GB) 1515716.7
Sep. 4, 2015 (GB) 1515718.3
Apr. 1, 2016 (GB) 1605583.2
May 17, 2016 (GB) 1608660.5

(51) Int. Cl. A61K 31/517; A61P 35/00; A61K 9/00; C07K 16/32; A61K 39/395; A61K 47/68; A61K 31/537; A61K 33/243; A61K 31/7068; A61K 31/513; A61K 31/519

(52) U.S. Cl. CPC A61K 31/517; A61P 35/00; A61K 9/0053; C07K 16/32; A61K 39/39558; A61K 47/6803; C07K 2317/24; A61K 31/537; A61K 33/243; A61K 31/7068; A61K 31/513; A61K 31/519; A61K 47/6855

(57) ABSTRACT

The present disclosure provides a method of treating a biliary duct cancer, such as cholangiocarcinoma, by administering a therapeutically effective amount of a compound of formula (I), in particular Varlitinib, an enantiomer thereof or a pharmaceutically acceptable salt of any one of the same.

The present disclosure relates to a therapy, for example a monotherapy or combination therapy comprising a type I tyrosine kinase inhibitor for the treatment of biliary cancer, such as cholangiocarcinoma.

### BACKGROUND

There are many cancers that are difficult to treat and although therapy is available, there appears to exist or to come into existence, a degree of resistance to the therapy. Primary resistance may occur in that cancer does not responsive to treatment from the outset. Secondary or acquired resistance also occurs quite frequently, which means that a therapy to which the patient seems to respond, at a certain time, loses its efficacy.

There are numerous reasons for resistance, for example some cancers are discovered at a late stage and/or a simply not responsive to treatment.

Mechanisms by which cancers avoid the therapeutic effect of therapy include but are not limited to:
i) mutations which render the cancer less vulnerable to the treatment (eg mutation of the site of action of the therapy),
ii) active transportation of the drug out of the tumor, for example by p-glycolation,
iii) building up physical defences, for example stroma which inhibit certain immune responses, and
iv) certain cancers develop paths to repair damage caused by some anti-cancer therapies.

Tumor heterogeneity may also contribute to resistance, where small subpopulations of cells may acquire or stochastically already possess some of the features enabling them to emerge under selective drug pressure. This is a problem that many patients with cancer encounter, and it obviously limits the therapeutic alternatives that are effective and worsens the prognosis.

Cancer therapy guidelines describe the sequence of therapies, which are recommended and in which sequence, so that if a patients show disease progression on the first therapy (“first line”), then a next therapy (“second line”) is recommended, and so on. These therapy recommendations are based on available scientific data and experience, and illustrate that resistance to one therapy does not exclude that another therapy may be effective and prolong life or shrink tumor. At a late stage cancers do not respond and no more avenues of therapy exist, and are thus judged as completely therapy refractory, unless new therapies can be found which are effective.

Cholangiocarcinoma is a prime example of both primary and secondary resistance and is considered to be an incurable and a rapidly lethal malignancy unless both the primary tumor and any metastases can be fully resected (removed surgically). No curative treatment exists for cholangiocarcinoma except surgery. Unfortunately, most patients have advanced stage disease which is inoperable at the time of diagnosis. Patients with cholangiocarcinoma are generally managed—though never cured—with chemotherapy, radiation therapy, and other palliative care measures. These are also used as adjuvant therapies (i.e. post-surgery) in cases where resection has apparently been successful (or nearly so).

In the western hemisphere cholangiocarcinoma is a relatively rare neoplasm that is classified as an adenocarcinoma (a cancer that forms glands or secretes significant amounts of mucins). It has an annual incidence rate of 1-2 cases per 100,000 in the Western world. However, rates of cholangiocarcinoma have been rising worldwide over the past several decades. Furthermore the incidence is higher in Asian countries where it is recognized as a significant problem.

Thus there a huge clinical need for improved therapies to address the lack effective treatment options for patients with biliary cancer.

(R)—N4-[3-Chloro-4-(thiazol-2-ylmethoxy)-phenyl]-N6-(4-methyl-4,5,-dihydro-oxazol-2-yl)-quinazoline-4,6-diamine (Varlitinib Example 52 disclosed in WO2005/016346), is a small-molecule pan-HER inhibitor. It has been tested as a monotherapy in phase I clinical trials of gastric cancer patients. 23 patients, who had previously failed on one or more rounds of chemotherapy, and where eligible for trastuzumab, each received 500 mg of Varlitinib orally twice daily (BID) as monotherapy for 28 days. Tumour biopsies taken before and after treatment were analysed using immunohistochemistry. Signs of clinical activity included downregulation of signalling pathways responsible for cell proliferation, and a reduction in cell survival and cell proliferation in gastric tumours that were either co-expressing EGFR and HER2 or that were HER2 amplified.

In further unpublished clinical work some of the cholangiocarcinoma patients who had previously had several lines of therapy which had failed at some stage were given Varlitinib monotherapy or Varlitinib in combination with chemotherapy. Varlitinib monotherapy/combination therapy showed a surprising level of efficacy in these patients. In particular, recent clinical trials have demonstrated that combination therapies involving Varlitinib in combination with capecitabine or in combination with gemcitabine and cisplatin may be particularly suitable for treating biliary duct cancer, such as cholangiocarcinoma. In addition Varlitinib appears to be efficacious and able to overcome both primary and secondary resistance in biliary duct cancer.

### SUMMARY OF THE DISCLOSURE

Thus, there is provided a method of treating a biliary duct cancer by administering a therapeutically effective amount of a compound of formula (I):

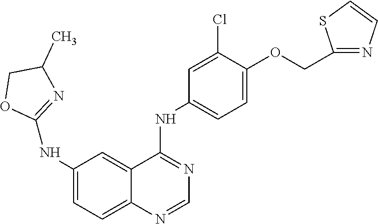 [SMILES: CC1COC(Nc2ccc3ncnc(Nc4ccc(OCc5nccs5)c(Cl)c4)c3c2)=N1]

an enantiomer thereof or a pharmaceutically acceptable salt of any one of same.

Thus in one embodiment there is provided a compound of formula (I) for use in the treatment of biliary duct cancer.

Also provided is use of a compound of formula (I) in the manufacture of a medicament for the treatment of biliary duct cancer.

In one embodiment the biliary duct cancer is selected from the group consisting of cholangiocarcinoma, gall bladder cancer and a combination thereof.

In one embodiment there is provided a method of treating a cholangiocarcinoma patient by administering a therapeutically effective amount of a compound of formula (I) an enantiomer thereof or a pharmaceutically acceptable salt of any one of the same.

In one aspect there is provided use of a compound of formula (I) an enantiomer thereof or a pharmaceutically acceptable salt of any one of the same in the treatment of cholangiocarcinoma.

Also provided is a compound of formula (I) an enantiomer thereof or a pharmaceutically acceptable salt of any one of the same for use in the treatment of cholangiocarcinoma.

In one aspect there provided use of a compound of formula (I) an enantiomer thereof or a pharmaceutically acceptable salt thereof for the manufacture of a medicament for the treatment of cholangiocarcinoma.

In one embodiment the cholangiocarcinoma is intrahepatic.

In one embodiment the cholangiocarcinoma is extrahepatic.

In one embodiment the biliary duct cancer is in a location selected from intrahepatic bile ducts, left hepatic duct, right hepatic duct, common hepatic duct, cystic duct, common bile duct, Ampulla of Vater and combinations thereof.

In one embodiment the biliary duct cancer is in an intrahepatic bile duct.

In one embodiment the biliary duct cancer is in a left hepatic duct.

In one embodiment the biliary duct cancer is in a right hepatic duct.

In one embodiment the biliary duct cancer is in a common hepatic duct.

In one embodiment the biliary duct cancer is in a cystic duct.

In one embodiment the biliary duct cancer is in a common bile duct.

In one embodiment the biliary duct cancer is in an Ampulla of Vater.

In one embodiment the biliary duct cancer is a cancer of the Papilla of Vater.

There is also provided a method of treating a gallbladder cancer patient by administering a therapeutically effective amount of a compound of formula (I) an enantiomer thereof or a pharmaceutically acceptable salt of any one of the same.

Thus in one aspect there is provided use of a compound of formula (I) an enantiomer thereof or a pharmaceutically acceptable salt of any one of the same in the treatment of gallbladder cancer.

Also provided is a compound of formula (I) an enantiomer thereof or a pharmaceutically acceptable salt of any one of the same for use in the treatment of gallbladder cancer.

In one aspect there provided use of a compound of formula (I) an enantiomer thereof or a pharmaceutically acceptable salt of any one of the same for the manufacture of a medicament for the treatment of gallbladder cancer.

In one embodiment the cancer is a metastatic form of a cancer disclosed herein.

In one embodiment the cancer according the present disclosure has not metastasized.

In one embodiment the compound of formula (I) is (R)—N4-[3-Chloro-4-(thiazol-2-ylmethoxy)-phenyl]-N6-(4-methyl-4, 5,-dihydro-oxazol-2-yl)-quinazoline-4,6-diamine:

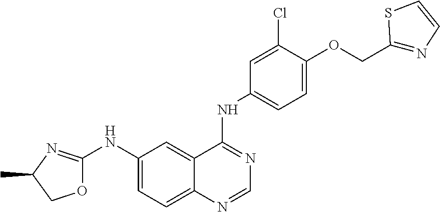 [SMILES: C[C@@H]1COC(Nc2ccc3ncnc(Nc4ccc(OCc5nccs5)c(Cl)c4)c3c2)=N1]

(Varlitinib) or a pharmaceutically acceptable salt thereof or a pro-drug thereof.

In one embodiment (R)—N4-[3-Chloro-4-(thiazol-2-ylmethoxy)-phenyl]-N6-(4-methyl-4, 5,-dihydro-oxazol-2-yl)-quinazoline-4,6-diamine is employed/administered as the free base (also referred to herein as Varlitinib).

Varlitinib at an appropriate dose is capable of inhibiting HER1, HER2 and HER4 directly and thought to be capable of inhibiting HER3 indirectly.

In one embodiment the compound of formula (I), such as Varlitinib at least inhibits the activity of HER1 and HER2, HER1 and HER4 or HER2 and HER4.

In one embodiment the compound of formula (I) at least inhibits the activity of HER1, HER2 and HER4, for example directly inhibits the activity of HER1, HER2 and HER4.

In one embodiment the compound of formula (I) inhibits the activity of HER1, HER2, HER3 and HER4, for example directly inhibits the activity of HER1, HER2, and HER4, and indirectly inhibits the activity of HER3.

In one embodiment the compound of formula (I) an enantiomer thereof or a pharmaceutically acceptable salt thereof is employed as a monotherapy, for example first line therapy or second line therapy, such as a first line monotherapy.

In one embodiment the compound of formula (I), an enantiomer thereof or a pharmaceutically acceptable salt thereof is employed in a combination therapy, for example in combination with a chemotherapy and/or a biological therapeutic, in particular as a first line therapy or a second line therapy.

In one embodiment the compound of formula (I), such as Varlitinib, is employed in combination with capecitabine.

In one embodiment each dose of capecitabine is 500-1500 mg/m², such as 500, 600, 700, 800, 900, 1000, 1100, 1200, 1300, 1400 or 1500 mg/m².

In one embodiment each dose of capecitabine is 1000 mg/m².

In one embodiment each dose of capecitabine is administered bi-daily.

In one embodiment each dose of capecitabine is 1000 mg/m² administered bi-daily.

In one embodiment the compound of formula (I), such as Varlitinib, is employed in combination with gemcitabine.

In one embodiment the compound of formula (I), such as Varlitinib, is employed in combination with cisplatin.

In one embodiment the compound of formula (I), such as Varlitinib, is employed in combination with gemcitabine and cisplatin.

In one embodiment each dose of gemcitabine is 500-1500 mg/m², such as 500, 600, 700, 800, 900, 1000, 1100, 1200, 1300, 1400 or 1500 mg/m².

In one embodiment each dose of gemcitabine is 1000 mg/m².

In one embodiment each dose of cisplatin is 5 to 50 mg/m², such as 5, 10, 15, 20, 25, 30, 35, 40, 45, 50 mg/m².

In one embodiment each dose of cisplatin is 25 mg/m².

In one embodiment each dose of gemcitabine is 1000 mg/m² and cisplatin is 25 mg/m².

In one embodiment each dose of cisplatin is administered first, followed by each dose of gemcitabine, such as a dose of 25 mg/m² cisplatin, followed by a dose of 1000 mg/m² gemcitabine

In one embodiment each dose of gemcitabine and cisplatin is administered once every 7 days.

In one embodiment each dose of gemcitabine and cisplatin is administered on Days 1 and 8, every 3 weeks.

In one embodiment a dose of 25 mg/m² cisplatin, followed by a dose of 1000 mg/m² gemcitabine is administered on Days 1 and 8, every 3 weeks.

In one embodiment the compound of formula (I), such as Varlitinib, is employed in combination with at least one further HER inhibitor, for example a combination of Varlitinib and Herceptin (trastuzumab) and/or pertuzumab. Surprisingly a combination of Varlitinib and Herceptin showed more therapeutic activity than either entity alone.

In one embodiment the compound of formula (I) such as Varlitinib is employed in combination with ado-trastuzuma-emtansine.

In one embodiment there is provided a method of treating biliary duct cancer comprising:

a) administering a therapeutically effective amount of a compound of formula (I):

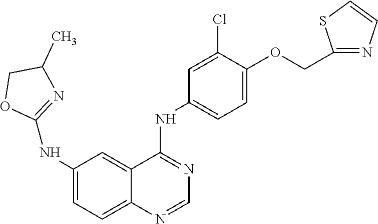 [SMILES: CC1COC(Nc2ccc3ncnc(Nc4ccc(OCc5nccs5)c(Cl)c4)c3c2)=N1]

an enantiomer thereof or a pharmaceutically acceptable salt of any one of the same, and

b) administering a chemotherapeutic agent or a combination of chemotherapeutic agents.

In one embodiment the compound of formula (I) an enantiomer thereof or a pharmaceutically acceptable salt thereof is employed as a second line monotherapy.

Surprisingly in a second line monotherapy trial employing Varlitinib in the treatment of cholangiocarcinoma patients showed a significant reduction in CA19-9 levels. CA19-9 is a marker employed in the management of cholangiocarcinoma. In a one patient a 90% reduction was seen in CA19-9 within one month of initiating treatment with Varlitinib.

Thus in one embodiment a patient has a 10, 20, 30, 40, 50, 60, 70, 80 or 90% decrease in CA19-9 level whilst on the therapy according to the present disclosure, wherein the level is decreased relative to the level of CA19-9 before initiation of said therapy.

In one embodiment the compound of formula (I), such as Varlitinib, is employed in a second line therapy together with a chemotherapy agent or chemotherapy regimen, for example gemcitabine, capecitabine, 5-FU, FOLFOX, a platin, such as cisplatin or oxaliplatin, and a combination thereof.

In one embodiment the compound of formula (I), such as Varlitinib, is administered orally.

In one embodiment the compound of formula (I), such as Varlitinib, is administered at a dose in the range 100 mg to 900 mg on each occasion, in particular 200 mg, 300 mg, 400 mg or 500 mg each dose, such as 400 mg, for example administered once or twice daily, such as twice daily.

In one embodiment the compound of formula (I), such as Varlitinib, is administered at a dose of 200 mg or 300 mg, for example twice daily (bi-daily).

In one embodiment the compounds of formula (I), such as Varlitinib, is administered at a dose of 300 mg, for example twice daily (bi-daily).

In one embodiment the compound of formula (I), such as Varlitinib, is administered at a dose of 200 mg, for example twice daily (bi-daily).

In one embodiment the compound of formula (I), such as Varlitinib, is administered at a dose of 200 mg or 300 mg twice daily (bi-daily), such as 300 mg twice daily, in a combination therapy with capecitabine.

In one embodiment the compound of formula (I), such as Varlitinib, is administered at a dose of 200 mg or 300 mg twice daily (bi-daily), such as 300 mg twice daily, in a combination therapy with capecitabine, wherein each dose of capecitabine is 1000 mg/m².

In one embodiment the compound of formula (I), such as Varlitinib, is administered at a dose of 200 mg or 300 mg twice daily (bi-daily), such as 300 mg twice daily, in a combination therapy with capecitabine, wherein each dose of capecitabine is 1000 mg/m² and is administered bi-daily, for example twice daily for 2 weeks, such as twice daily for 2 weeks followed by a 1 week rest period.

In one embodiment the compound of formula (I), such as Varlitinib, is administered at a dose of 200 mg or 300 mg twice daily (bi-daily), such as 300 mg twice daily, in a combination therapy with capecitabine, wherein each dose of capecitabine is 1000 mg/m² and is administered bi-daily for 2 weeks, such as twice daily for 2 weeks followed by a 1 week rest period.

In one embodiment the compound of formula (I), such as Varlitinib, is administered at a dose of 200 mg or 300 mg twice daily (bi-daily), such as 200 mg twice daily, in a combination therapy with gemcitabine and cisplatin.

In one embodiment the compound of formula (I), such as Varlitinib, is administered at a dose of 200 mg or 300 mg twice daily (bi-daily), such as 200 mg twice daily, in a combination therapy with gemcitabine and cisplatin, wherein each dose of gemcitabine is 1000 mg/m².

In one embodiment the compound of formula (I), such as Varlitinib, is administered at a dose of 200 mg or 300 mg twice daily (bi-daily), such as 200 mg twice daily, in a combination therapy with gemcitabine and cisplatin, wherein each dose of cisplatin is 25 mg/m².

In one embodiment the compound of formula (I), such as Varlitinib, is administered at a dose of 200 mg or 300 mg twice daily (bi-daily), such as 200 mg twice daily, in a combination therapy with gemcitabine and cisplatin, wherein each dose of gemcitabine is 1000 mg/m² and cisplatin is 25 mg/m², for example wherein the gemcitabine is administered after cisplatin.

In one embodiment the compound of formula (I), such as Varlitinib, is administered at a dose of 200 mg or 300 mg twice daily (bi-daily), such as 200 mg twice daily, in a combination therapy with gemcitabine and cisplatin, wherein each dose of gemcitabine is 1000 mg/m² and cisplatin is 25 mg/m², wherein the gemcitabine is administered after cisplatin, and wherein the gemcitabine is administered after cisplatin on Days 1 and 8, such as on Days 1 and 8 every 3 weeks.

In one embodiment the cisplatin and/or gemcitabine is administered parenterally, for example intravenously (IV) or by infusion. In one embodiment the cisplatin is administered by IV for 0.25 to 3 hours, such as 2 hours. In one embodiment the gemcitabine is administered by 0.25 to 3 hour infusion, such as 0.5 hrs. In one embodiment the cisplatin is administered at 25 mg/m², IV for 2 hours and the gemcitabine is administered at 1000 mg/m² for 0.5 hrs.

In one embodiment the compound of formula (I), such as Varlitinib, is administered for 28 days, referred to herein as a 28 day treatment cycle.

In one embodiment the compound of formula (I) is administered as pharmaceutical formulation comprising one or more pharmaceutically acceptable excipients.

In one embodiment the compound of formula (I) or a formulation comprising the same is administered orally, for example as tablet or capsule.

In one embodiment the target patient population is at least HER1 (EGFR) positive.

In one embodiment the target patient population is EGFR and HER2 positive.

In one embodiment the target patient population is at least HER1 (EGFR) positive.

In one embodiment the target patient population are HER2 amplified.

In one embodiment the patient has over-expression of 2 or more of the HER 1, 2, 3, 4 receptors, for example over expression of HER 1 & 2; 1 & 3; 1 & 4; 2 & 3 2 & 4; 1, 2 & 3; 1, 2 & 4; 2, 3 &4; or 1, 2, 3 &4.

In one embodiment the treatment is adjuvant therapy, for example after surgery or after chemotherapy.

In one embodiment the treatment is neoadjuvant therapy, for example before surgery, in particular to shrink the tumour or tumours.

In one embodiment the cancer is a tumour, in particular a solid tumour. In one embodiment the treatment according to the present disclosure is suitable for the treatment of secondary tumours. In one embodiment the cancer is metastatic cancer. In one embodiment the cancer is naïve advanced. In one embodiment the treatment according to the present disclosure is suitable for the treatment of primary cancer and metastases. In one embodiment the treatment according to the present disclosure is suitable for the treatment of secondary cancer and metastases. In one embodiment the treatment according to the present disclosure is suitable for the treatment of primary cancer, secondary cancer and metastases.

In one embodiment the treatment according to the present disclosure is suitable for the treatment of cancerous cells in a lymph node, for a cancer of the present disclosure.

In one embodiment the patient is a human.

### DETAILED DISCLOSURE

Biliary duct cancer (also referred to as biliary cancer or biliary tract cancer) as employed herein refers to cancer which starts in the bile ducts and includes cholangiocarcinoma and gallbladder cancer.

Cholangiocarcinoma as referred to herein is a form of cancer that is composed of mutated epithelial cells (or cells showing characteristics of epithelial differentiation) that originate in the bile ducts which drain bile from the liver into the small intestine, but not including gallbladder cancer.

General guidelines for operability include:
Absence of lymph node or liver metastases
Absence of involvement of the portal vein
Absence of direct invasion of adjacent organs
Absence of widespread metastatic disease

Gallbladder cancer as employed herein cancer which starts in the gallbladder. The following stages are used for gallbladder cancer:
Stage 0 (carcinoma in situ): Abnormal cells are found in the inner (mucosal) layer of the gallbladder; these abnormal cells may become cancer and spread into nearby normal tissue,
Stage I: Cancer has formed and has spread beyond the inner (mucosal) layer to a layer of tissue with blood vessels or to the muscle layer,
Stage II: Cancer has spread beyond the muscle layer to the connective tissue around the muscle,
Stage IIIA: Cancer has spread through the thin layers of tissue that cover the gallbladder and/or to the liver and/or to one nearby organ (e.g., stomach, small intestine, colon, pancreas, or bile ducts outside the liver),
Stage IIIB: Cancer has spread to nearby lymph nodes and beyond the inner layer of the gallbladder to a layer of tissue with blood vessels or to the muscle layer; or beyond the muscle layer to the connective tissue around the muscle; or through the thin layers of tissue that cover the gallbladder and/or to the liver and/or to one nearby organ,
Stage IVA: Cancer has spread to a main blood vessel of the liver or to 2 or more nearby organs or areas other than the liver. Cancer may have spread to nearby lymph nodes.
Stage IVB: Cancer has spread to either lymph nodes along large arteries in the abdomen and/or near the lower part of the backbone or to organs or areas far away from the gallbladder.

In one embodiment the treatment of the present disclosure is neo-adjuvant therapy, for example to shrink the tumour/carcinoma before surgery to remove the cancerous tissue or before chemotherapy to improve the chances of success of the latter or to reduce the severity of the treatment required.

In one embodiment the treatment of the present disclosure is adjuvant therapy, for example following surgery to remove the cancerous tissue.

In one embodiment the treatment of the present disclosure is adjuvant therapy, for example following chemotherapy.

In patients where not all the cancerous tissue is removed by surgery then the patient may benefit from adjuvant therapy which is monotherapy employing a compound of formula (I), such as Varlitinib.

In patients where not all the cancerous tissue is removed by surgery then the patient may benefit from combination adjuvant therapy comprising a compound of formula (I) and chemotherapy or radiotherapy.

First line therapy as employed herein is the first therapy employed for the treatment of the cancer and in some instances the first line therapy may be neo-adjuvant therapy, in this context surgery will generally be considered a treatment.

Second line therapy as employed herein is treatment following first line therapy and may be adjuvant therapy. Thus in the context of the present specification second line therapy is simply therapy other than first line therapy and includes, third line therapy, fourth line therapy etc.

Monotherapy as employed herein is wherein the compound of formula (I) an enantiomer thereof and/or a pharmaceutically acceptable salt thereof, is the only active agent being administered to the patient for the treatment of cancer.

Combination therapy as employed herein refers to wherein the compound of formula (I) an enantiomer thereof or a pharmaceutically acceptable salt thereof is employed for the treatment of the cancer in conjunction with one or more further anticancer treatments, for example where the treatment regimens for the two or more active anticancer agents overlap or where the two or more anticancer agents are administered concomitantly.

In one embodiment the combination therapy according to the present disclosure comprises a RON inhibitor, for example as disclosed WO2008/058229, incorporated herein by reference.

In one embodiment the combination therapy comprises a checkpoint inhibitor, such as a CTLA4 inhibitor, a PD-1 inhibitor or a PD-L1 inhibitor, in particular an antibody or binding fragment thereof.

Examples of pharmaceutically acceptable salts include but are not limited to acid addition salts of strong mineral acids such as HCl and HBr salts and addition salts of strong organic acids, such as a methansulfonic acid salt, tosylates, furoates and the like, including di, tri salts thereof, such as ditosylates.

Analysis of patients to profile their cancer, for example to establish if their cancer is EGFR and HER2 positive is known and is routine in the art Establishing if a cancer is HER2 amplified is also routine in the art.

### Chemotherapeutic Agents

Chemotherapeutic agent and chemotherapy or cytotoxic agent are employed interchangeably herein unless the context indicates otherwise.

Chemotherapy as employed herein is intended to refer to specific antineoplastic chemical agents or drugs that are “selectively” destructive to malignant cells and tissues, for example alkylating agents, antimetabolites including thymidylate synthase inhibitors, anthracyclines, anti-microtubule agents including plant alkaloids, taxanes, topoisomerase inhibitors, parp inhibitors and other antitumour agents. Selectively in this context is used loosely because of course many of these agents have serious side effects.

The preferred dose may be chosen by the practitioner, based on the nature of the cancer being treated.

Examples of alkylating agents, which may be employed in the method of the present disclosure include an alkylating agent, nitrogen mustards, nitrosoureas, tetrazines, aziridines, platins and derivatives, and non-classical alkylating agents.

Examples of platinum containing chemotherapeutic agents (also referred to as platins), include cisplatin, carboplatin, oxaliplatin, satraplatin, picoplatin, nedaplatin, triplatin and lipoplatin (a liposomal version of cisplatin), in particular cisplatin, carboplatin and oxaliplatin.

The dose for cisplatin ranges from about 20 to about 270 mg/m² depending on the exact cancer. Often the dose is in the range about 70 to about 100 mg/m².

Nitrogen mustards include mechlorethamine, cyclophosphamide, melphalan, chlorambucil, ifosfamide and busulfan.

Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide.

Aziridines include thiotepa, mytomycin and diaziquone (AZQ).

Examples of antimetabolites, which may be employed in the method of the present disclosure, include anti-folates (for example methotrexate and pemetrexed), purine analogues (for example thiopurines, such as azathiopurine, mercaptopurine, thiopurine, fludarabine (including the phosphate form), pentostatin and cladribine), pyrimidine analogues (for example fluoropyrimidines, such as 5-fluorouracil (5-FU) and prodrugs thereof such as capecitabine [Xeloda®]), floxuridine, gemcitabine, cytarabine, decitabine, raltitrexed (tomudex) hydrochloride, cladribine and 6-azauracil.

Examples of anthracyclines, which may be employed in the method of the present disclosure, include daunorubicin (Daunomycin), daunorubicin (liposomal), doxorubicin (Adriamycin), doxorubicin (liposomal), epirubicin, idarubicin, valrubicin currently are used only to treat bladder cancer and mitoxantrone an anthracycline analog, in particular doxorubicin.

Examples of anti-microtubule agents, which may be employed in the method of the present disclosure, include vinca alkaloids and taxanes.

Vinca alkaloids include completely natural chemicals for example vincristine and vinblastine and also semi-synthetic vinca alkaloids, for example vinorelbine, vindesine, and vinflunine Taxanes include paclitaxel, docetaxel, abraxane, carbazitaxel and derivatives of thereof.

Derivatives of taxanes as employed herein includes reformulations of taxanes like taxol, for example in a micelluar formulaitons, derivatives also include chemical derivatives wherein synthetic chemistry is employed to modify a starting material which is a taxane.

Topoisomerase inhibitors, which may be employed in a method of the present disclosure include type I topoisomerase inhibitors, type II topoisomerase inhibitors and type II topoisomerase poisons. Type I inhibitors include topotecan, irinotecan, indotecan and indimitecan. Type II inhibitors include genistein and ICRF 193 which has the following structure:

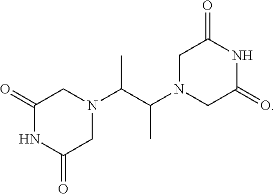 [SMILES: CC(C(C)N1CC(=O)NC(=O)C1)N1CC(=O)NC(=O)C1]

Type II poisons include amsacrine, etoposide, etoposide phosphate, teniposide and doxorubicin and fluoroquinolones.

In one embodiment the chemotherapeutic is a PARP inhibitor.

In one embodiment a combination of chemotherapeutic agents employed is, for example a platin and 5-FU or a prodrug thereof, for example cisplatin or oxaplatin and capecitabine or gemcitabine, such as FOLFOX.

In one embodiment the chemotherapy comprises a combination of chemotherapy agents, in particular cytotoxic chemotherapeutic agents.

In one embodiment the chemotherapy combination comprises capecitabine.

In one embodiment the chemotherapy combination comprises gemcitabine and cisplatin.

In one embodiment the chemotherapy combination comprises a platin, such as cisplatin and fluorouracil or capecitabine.

In one embodiment the chemotherapy combination is capecitabine and oxaliplatin (XELOX).

In one embodiment the chemotherapy is a combination of folinic acid and 5-FU, optionally in combination with oxaliplatin (FOLFOX).

In one embodiment the chemotherapy is a combination of folinic acid, 5-FU and irinotecan (FOLFIRI), optionally in combination with oxaliplatin (FOLFIRINOX). The regimen, for example includes: irinotecan (180 mg/m² IV over 90 minutes) concurrently with folinic acid (400 mg/m² [or 2×250 mg/m²] IV over 120 minutes); followed by fluorouracil (400-500 mg/m² IV bolus) then fluorouracil (2400-3000 mg/m² intravenous infusion over 46 hours). This cycle is typically repeated every two weeks. The dosages shown above may vary from cycle to cycle.

In one embodiment the chemotherapy combination employs a microtubule inhibitor, for example vincristine sulphate, epothilone A, N-[2-[(4-Hydroxyphenyl)amino]-3-pyridinyl]-4-methoxybenzenesulfonamide (ABT-751), a taxol derived chemotherapeutic agent, for example paclitaxel, abraxane, or docetaxel or a combination thereof.

In one embodiment the chemotherapy combination employs an mTor inhibitor.

Examples of mTor inhibitors include: everolimus (RAD001), WYE-354, KU-0063794, papamycin (Sirolimus), Temsirolimus, Deforolimus(MK-8669), AZD8055 and BEZ235(NVP-BEZ235).

In one embodiment the chemotherapy combination employs a MEK inhibitor. Examples of MEK inhibitors include: AS703026, CI-1040 (PD184352), AZD6244 (Selumetinib), PD318088, PD0325901, AZD8330, PD98059, U0126-EtOH, BIX 02189 or BIX 02188.

In one embodiment the chemotherapy combination employs an AKT inhibitor. Examples of AKT inhibitors include: MK-2206 and AT7867.

In one embodiment the combination employs an aurora kinase inhibitor. Examples of aurora kinase inhibitors include: Aurora A Inhibitor I, VX-680, AZD 1152-HQPA (Barasertib), SNS-314 Mesylate, PHA-680632, ZM-447439, CCT129202 and Hesperadin.

In one embodiment the chemotherapy combination employs a p38 inhibitor, for example as disclosed in WO2010/038086, such as N-[4-({4-[3-(3-tert-Butyl-1-p-tolyl-1H-pyrazol-5-yl)ureido]naphthalen-1-yloxy}methyl)pyridin-2-yl]-2-methoxyacetamide.

In one embodiment the combination employs a Bcl-2 inhibitor. Examples of Bcl-2 inhibitors include: obatoclax mesylate, ABT-737, ABT-263(navitoclax) and TW-37.

In one embodiment the chemotherapy combination comprises an antimetabolite such as capecitabine (xeloda), fludarabine phosphate, fludarabine (fludara), decitabine, raltitrexed (tomudex), gemcitabine hydrochloride and/or cladribine.

In one embodiment the chemotherapy combination comprises ganciclovir, which may assist in controlling immune responses and/or tumour vasculation.

In one embodiment after combination therapy a monotherapy comprising a compound of formula (I), such as Varlitinib (as defined herein including doses described above) is employed, for example a maintenance therapy.

“Comprising” in the context of the present specification is intended to mean “including”. Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The invention will now be described with reference to the following examples, which are merely illustrative and should not in any way be construed as limiting the scope of the present invention.

### BRIEF SUMMARY OF THE FIGURES

FIG. 1 Shows the position of bile ducts in the body

FIG. 2 Shows data for a cholangiocarcinoma patient receiving 400 mg of Varlitinib monotherapy bi-daily

FIG. 3 Shows data for a cholangiocarcinoma patient receiving 400 mg Varlitinib bi-daily in combination with chemotherapy.

FIG. 4 Shows data for a 56 year old male cholangiocarcinoma patient treated with Varlitinib 400 mg bi-daily and cisplatin/capecitabine chemotherapy

FIG. 5 Shows data for a 60 year old male cholangiocarcinoma patient treated with Varlitinib 500 mg bi-daily and cisplatin/capecitabine chemotherapy

FIG. 6a Shows change in a lesion of over time for individual cholangiocarcinoma patients receiving between 300 and 500 mg of Varlitinib bi-daily and chemotherapy in a Taiwanese clinical trial

FIG. 6b Shows change in a lesion of over time for individual cholangiocarcinoma patients receiving between 300 and 500 mg of Varlitinib bi-daily and chemotherapy in a Singapore clinical trial

FIG. 7a Shows the maximal % change in tumour size from baseline in individual cholangiocarcinoma patients receiving between 300-500 mg of Varlitinib bi-daily and chemotherapy in Taiwan clinical trial

FIG. 7b Shows the maximal % change in tumour size from baseline in individual cholangiocarcinoma patients receiving between 300-500 mg of Varlitinib bi-daily and chemotherapy in Taiwan clinical trial

FIG. 8a Shows the study scheme for the Phase 2A trial of Varlitinib plus capecitabine in Chinese patients with advanced or metastatic biliary tract cancer.

FIG. 8b Shows study design for the Phase 2A trial of Varlitinib plus capecitabine in Chinese patients with advanced or metastatic biliary tract cancer.
Key inclusion criteria include having failed gemcitabine-containing 1st line systemic treatment, and no evidence of biliary duct obstruction (unless controlled by local treatment or endoscopic or percutaneous decompression).
Treatment will continue until disease progression or unacceptable toxicity, withdrawal of consent, or death

FIG. 9a Shows the % of subject by gender of the patients.

FIG. 9b Shows the number of subjects by age.

FIG. 9c Shows the number of subjects by Eastern Cooperative Oncology Group scores (ECOG) at C1D1 (Day 1 of study treatment cycle 1).

FIG. 9d Shows the % of subject by primary tumor site.

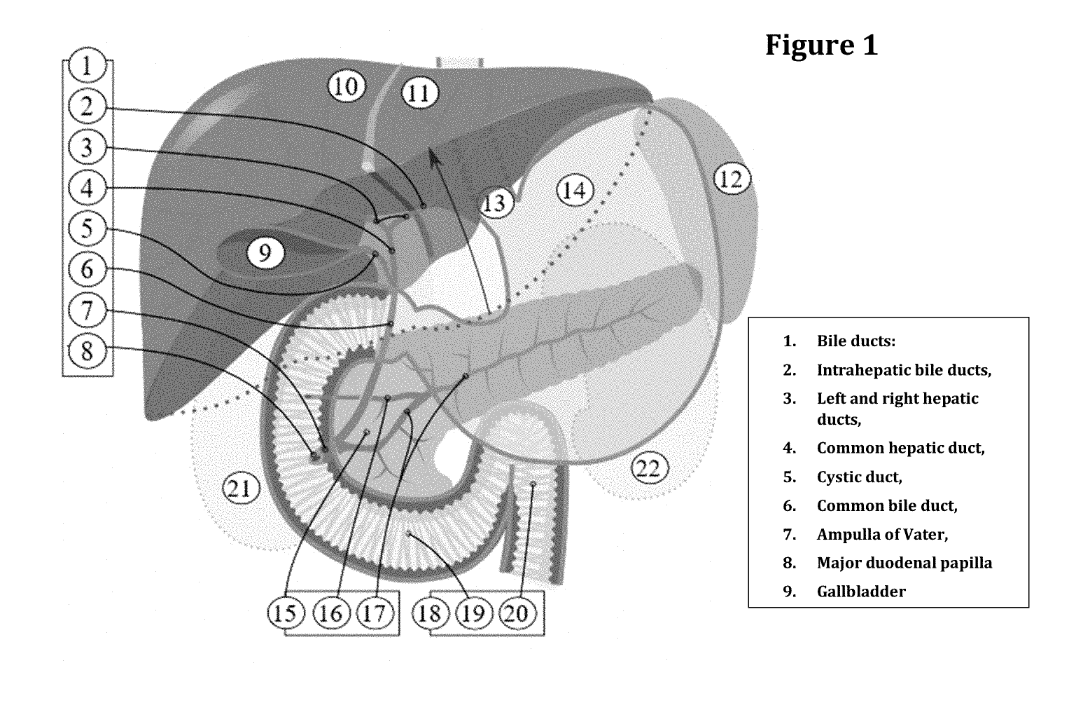

### EXAMPLES

### Example 1 Varlitinib 400 mg Bi-Daily Monotherapy

A 45 year-old male stage IV cholangiocarcinoma EGFR positive (3+) cancer patient had progressive disease following:
first line treatment with gemcitabine (partial remission), and
second line treatment with cisplatin and 5-FU.

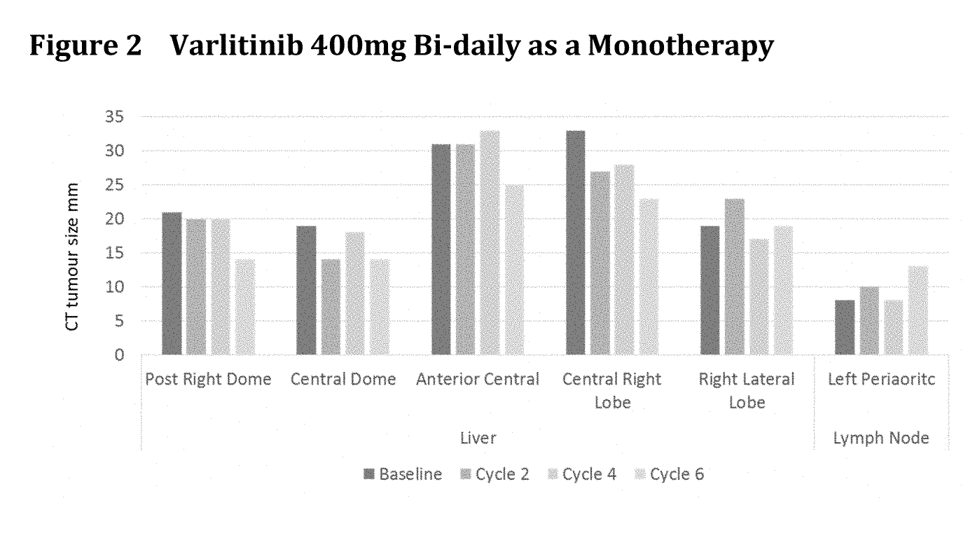

The results are shown in FIG. 2. After treatment cycle 6 with Varlitinib 400 mg bi-daily the liver tumours decreased in size up to 23% and the tumour marker CA 19-9 fell from ˜900 U/ml to ˜250 U/ml.

### Example 2 Varlitinib 400 mg Bi-Daily Monotherapy

A 58 year-old, male, stage IV, extra-hepatic cholangiocarcinoma, prior treatment:
Whipple
Radiotherapy
Gemzar/cisplatin for 6 months

For the first 6 cycles, the patient received Varlitinib 400 mg BID continuously with cisplatin (80 mg/m² every 3 weeks) and capecitabine (1000 mg/m2 BID, 2 weeks on, 1 week off). Image scan at the end of cycle 6 showed 85.77% reduction in tumour size. After cycle 6, the patient received varlitinib monotherapy and tumor scan at the end of cycle 8 showed 87% reduction. This patient showed partial remission for 24 weeks. However, at the end of cycle 10, image scan showed tumour enlarged to the extent that met criteria for disease progression based on RECIST (>20% increase against nadir), so the patient was withdrawn from this study. In summary, the patient received varlitinib with chemo for 6 cycles and varlitinib monotherapy for another 4 cycles (3 weeks per cycle).

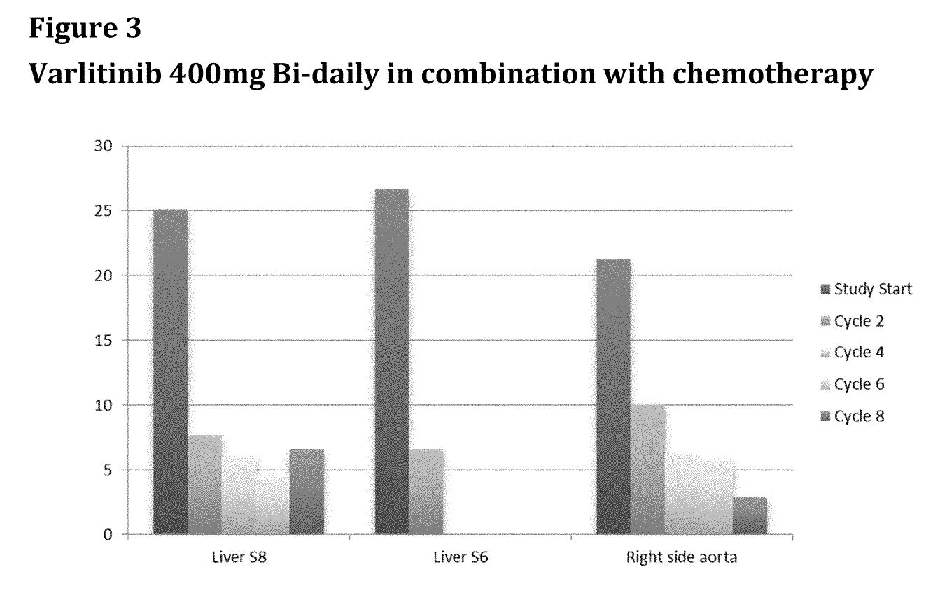

The results are shown in FIG. 3.

### Example 3 Treatment of Cholangiocarcinoma with Varlitinib 400 mg Bi-Daily Orally and Cisplatin/Capecitabine Combination Chemotherapy

A 56 year-old male with stage IV cholangiocarcinoma (3 lesions) had progressive disease following treatment with:
radiotherapy, and
gemcitabine (Gemzar®) and cisplatin—6 months.

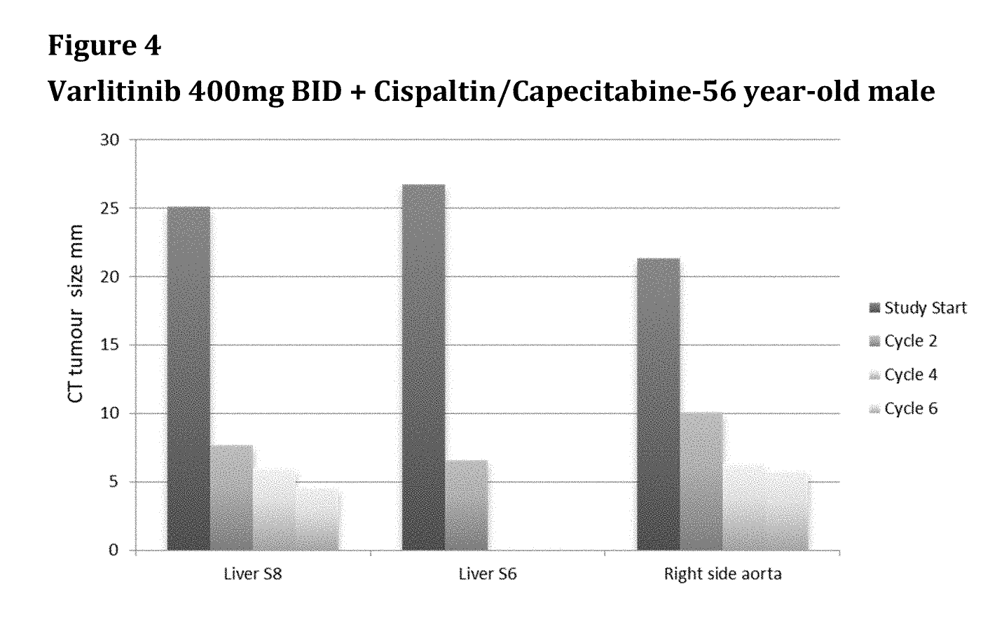

After treatment cycle 6 with Varlitinib 400 mg bi-daily, cisplatin and capecitabine the patient showed an 85.77% response, see FIG. 4. No dose limiting toxicity was observed in the first two treatment cycles. Varlitinib was well tolerated. The cisplatin and capecitabine regime was cisplatin 80 mg/m² IV infusion and capecitabine 1000 mg/m² orally twice daily for 14 days every 3 weeks.

### Example 4 Treatment of Cholangiocarcinoma with Varlitinib 500 mg Bi-Daily Orally and Cisplatin/Capecitabine Combination Chemotherapy

A 60 year-old male with stage IV cholangiocarcinoma had progressive disease following treatment with radiotherapy and bi-weekly 5-FU.

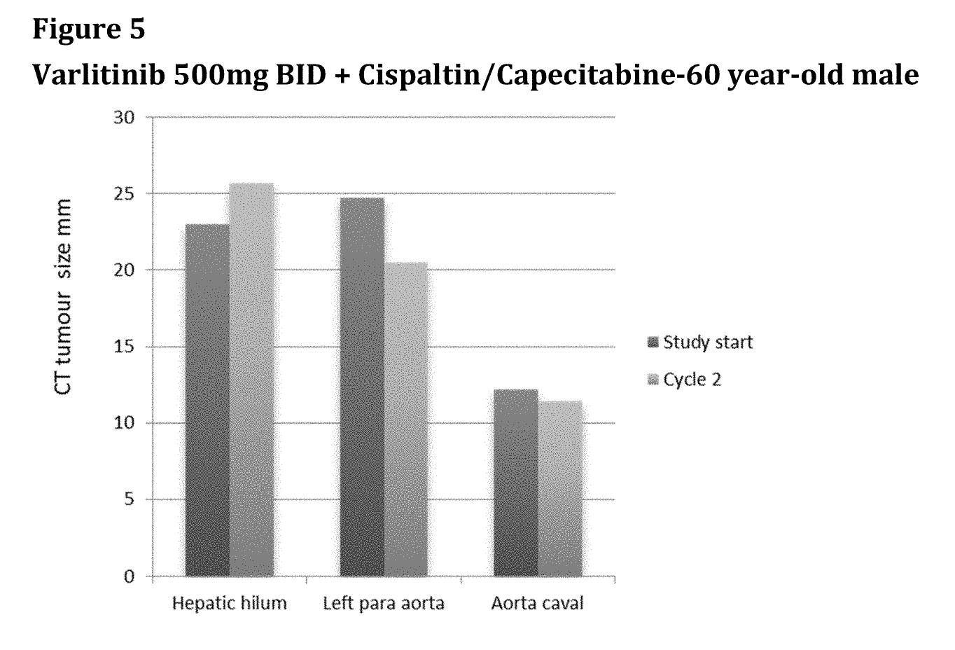

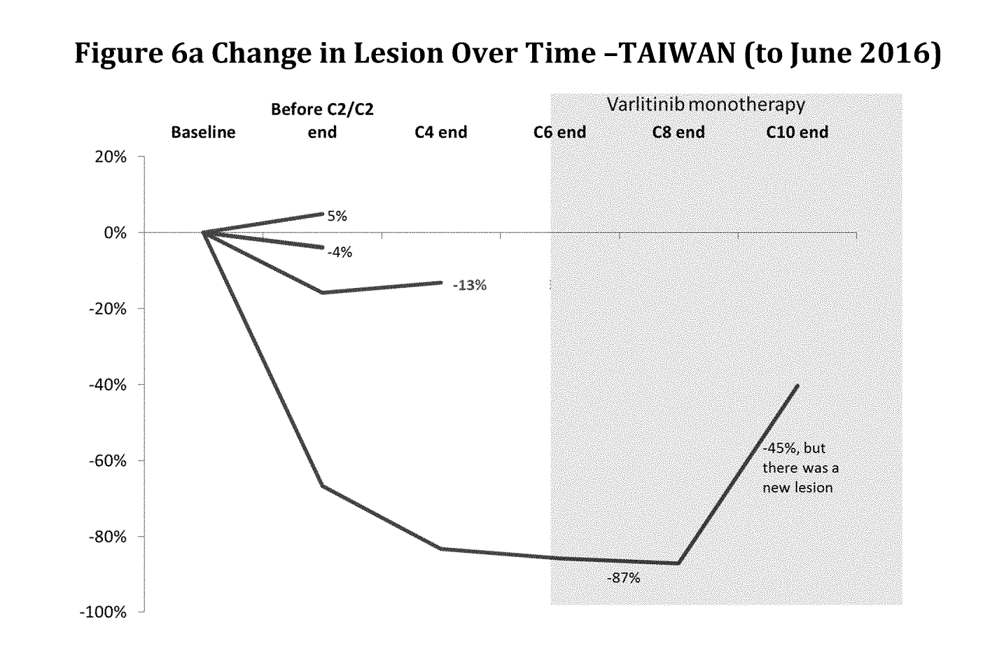

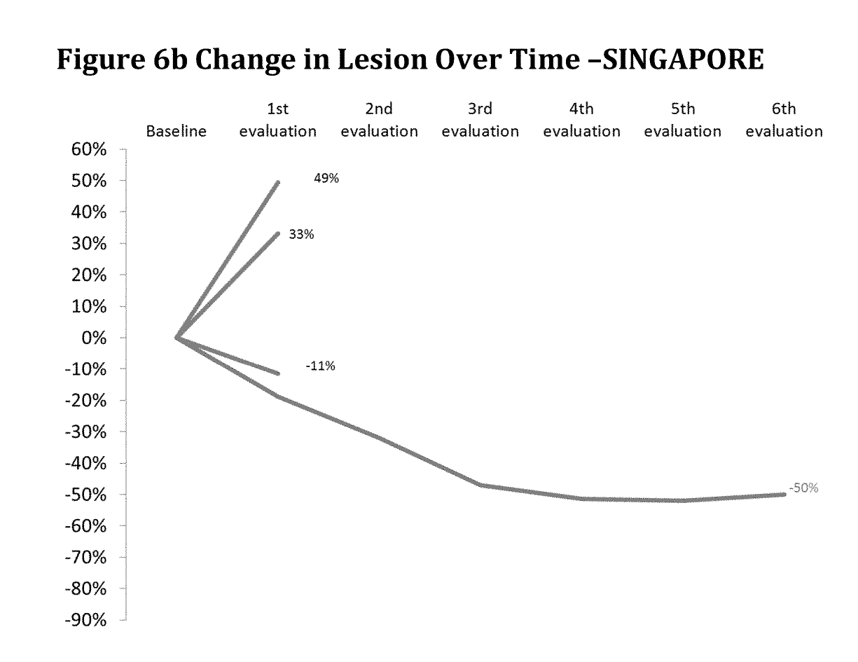

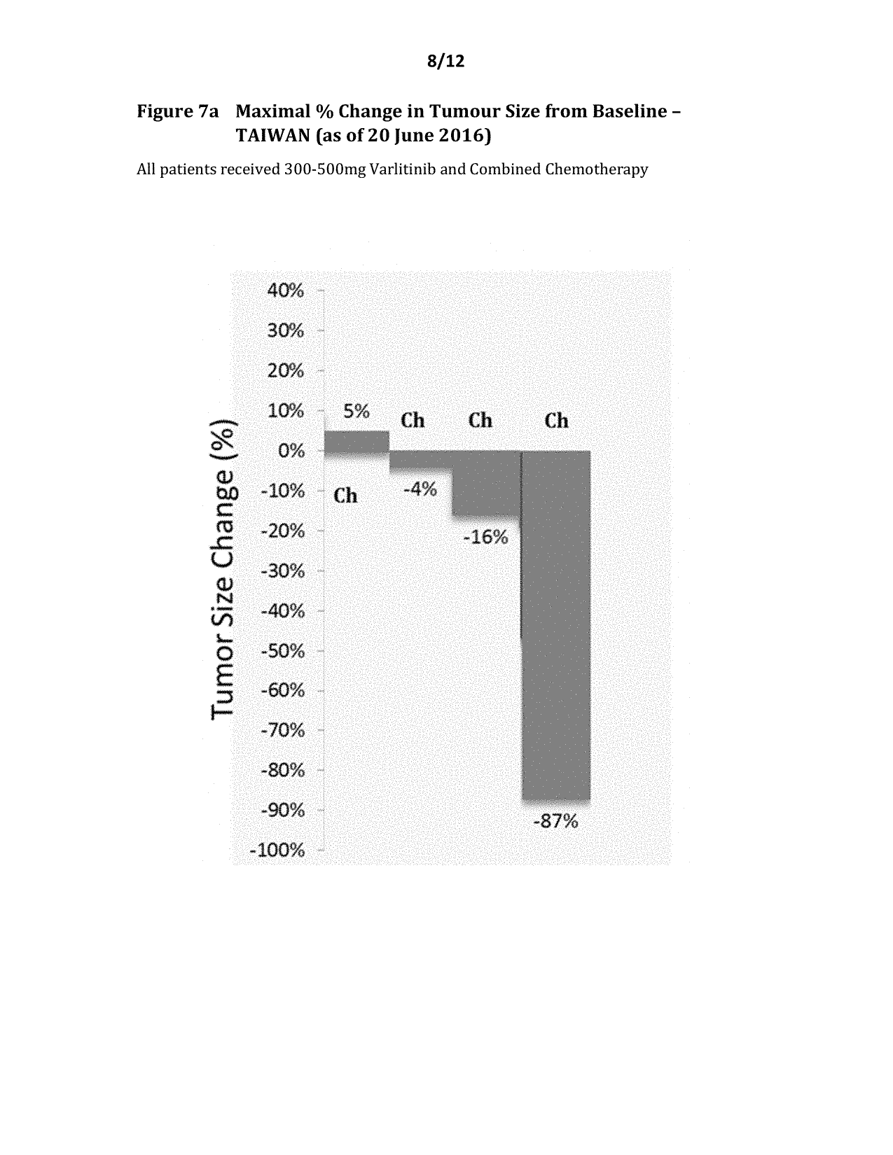

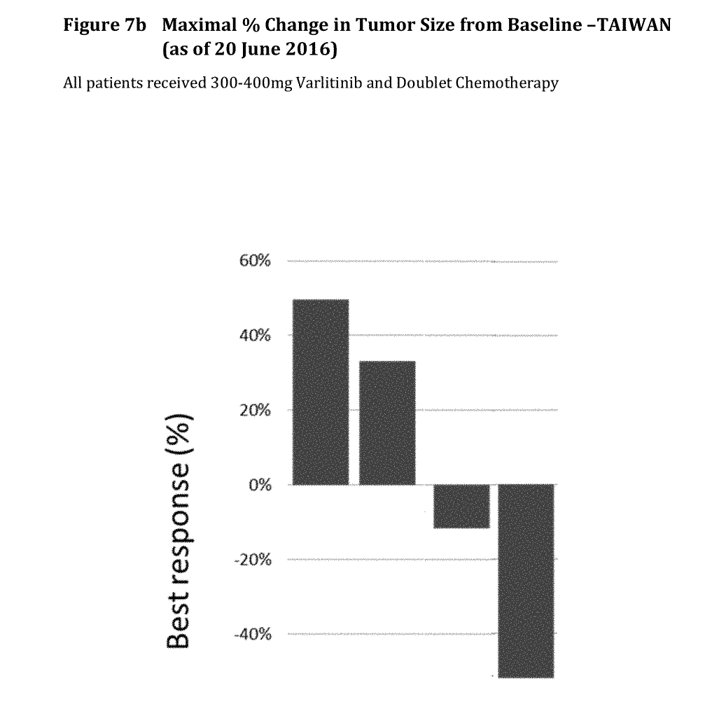

After treatment cycle 2 with Varlitinib orally 500 mg bi-daily, cisplatin and capecitabine the patient showed a 4% response, see FIG. 5. The cisplatin and capecitabine regime was cisplatin 80 mg/m² IV infusion and capecitabine 1000 mg/m² orally twice daily for 14 days every 3 weeks.

### Example 5 Treatment of Stage IV Cholangiocarcinoma and Metastatic Lymphadenopathy with Varlitinib 400 mg Bi-Daily Orally and Cisplatin/5FU Combination Chemotherapy

A 49 year-old male was diagnosed with stage IV cholangiocarcinoma and metastatic lymphadenopathy in January 2016. No prior surgery or treatment was given before initiating first line treatment of 300 mg BID Varlitinib combined with Cisplating/5-FU, 28-day treatment cycle.

Date of the first response 5 Mar. 2016, tumor C2 was reduced by 16%. Last response tumor C4 was reduced by 13% (17 May 2016). Patient current disease status is stable disease.

### Example 6 Treatment of the Intrahepatic Bile Duct Cholangiocarcinoma with 300 mg Valitinib and FOLFOX

A 51 year old female diagnosed in 2013 with intrahepatic bile duct cholangiocarcinoma, had received surgery in the form of left hemihepatectomy in 15 Jul. 2013. Prior therapies were gemcitabine and cisplatin (in the period 14 Aug. 2013 to 8 Jan. 2014) and repeated between 13 May 2015 and 1 Jul. 2015. With this treatment the status was progressive disease. The patient was given 9 cycles of a combination of Varlitinib 400 mg reduced to 300 mg BID and FOLFOX. This was followed with 7 cycles of Varlitinib monotherapy. The patient status is partial response with a reduction is tumor size of 50%.

### Example 7 Treatment of Stage IV Cholangiocarcinoma with Varlitinib 400 mg Bi-Daily Orally and Cisplatin/5FU Combination Chemotherapy

A 69 year-old female with stage IV cholangiocarcinoma newly diagnosed in 2014 received surgery (percutaneous transhepatic cholangial drainage and transcatheter anterior embolization), followed by adjuvant treatment of Varlitinib 400 mg BID combined with cisplatin s/5-FU 28-day cycle. After cycle 6 (14 Jul. 2015), only 400 mg BID Varlitinib was taken. Patient completed 10 cycles of monotherapy. At Cycle 11 Day 1, dose was reduced to 300 mg BID alternate days. First response was observed in cycle 2 of treatment 17 Mar. 2015. Progressive disease observed in cycle 16 of monotherapy 12 Apr. 2016. This patient demonstrated remarkable disease control on varlitinib monotherapy after 6 cycles of varlitinib in combination with chemotherapy.

### Example 8 Treatment of Stage IV Intrahepatic Cholangiocarcinoma with Varlitinib 400 mg Bi-Daily Orally and FOLFOX

A 51 year-old female who had a medical history of meningioma post excision with left craniotomy was diagnosed with intrahepatic cholangiocarcinoma, stage IV with metastasis to portacaval lymph nodes and liver in August 2014. She received left hemihepatectomy followed by 6 cycles of adjuvant gemcitabine and cisplatin. The patient's disease progressed in May 2015 and she received 1st line gemcitabine and cisplatin in metastatic setting, to which he did not respond. After disease progression on gemcitabine and cisplatin, she was enrolled into the ASLAN001-002SG study in August 2015 and received Varlitinib 400 mg BID in combination with mFOLFOX6 (2 weeks per cycle). To date (22 Aug. 2016), the patient has completed 9 cycles of Varlitinib and mFOLFOX6, as well as, 9 cycles of varlitinib monotherapy and the latest tumor assessment completed after cycle 18 continues to show partial response with 53% reduction in tumor size as the best response from baseline.

### Example 9 Treatment of Stage IV Intrahepatic Cholangiocarcinoma and Multiple Lymphadenopathies with Varlitinib 300 mg Bi-Daily Orally and Cisplatin/5FU Combination Chemotherapy 28 Day Cycle

A 64 year old female diagnosed with intrahepatic cholangiocarcinoma and multiple lymphadenopathies in May 2016 received Varlitinib combination therapy as the first line treatment.

The patient was given 300 mg BID Varlitinib combined with Cis/5-FU, 28-day cycle. The current status is the clinical trial is on-going.

### Example 10 Phase 2A, Single Arm, Multicenter Study of Varlitinib Plus Capecitabine in Chinese Patients with Advanced or Metastatic Biliary Tract Cancer (BTC)

This study is to assess the efficacy of varlitinib (300 mg twice daily in a 21-day cycle) plus capecitabine (1,000 mg/m² twice daily for 2 weeks followed by a 7-day rest period) in patients with advanced or metastatic BTC.

Primary Objective:
To assess the efficacy of varlitinib plus capecitabine in patients with advanced or metastatic biliary tract cancer who progressed on first line of systemic therapy as measured by Objective Response Rate (ORR) based on RECIST v1.1.

Secondary Objectives:
To evaluate the efficacy of varlitinib plus capecitabine, as measured by duration of confirmed response (DoR), progression-free survival (PFS), overall survival (OS) and disease control rate (DCR)
To assess the safety and tolerability of varlitinib when combined with capecitabine

Study Scheme

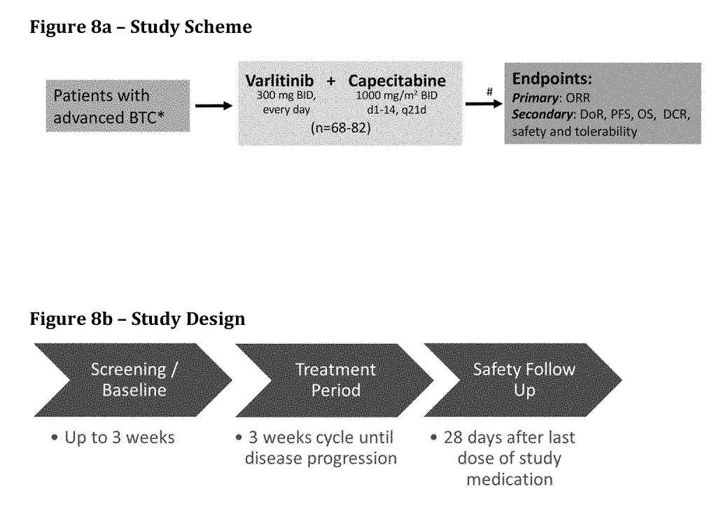

A phase 2A, single arm, multicentre study of varlitinib plus capecitabine in Chinese patients with advanced or metastatic biliary tract cancer who progressed on at least 1 line of systemic therapy. FIG. 8a shows details of the study scheme.

### Study Design

The Study duration is divided into 3 different stages. FIG. 8b shows a flow chart of the study design.
Screening period: 3 weeks
Treatment
   Varlitinib 300 mg BID every day+Capecitabine 1000 mg/m² BID for 2 weeks followed by a 1-week rest until disease progression, unacceptable toxicity, consent withdrawal or death
   Radiological imaging to assess disease status will be performed at baseline and every 6 weeks until disease progression to evaluate the efficacy of treatment according to RECIST Version 1.1
Safety follow up: 28 days after the last dose of study medication.

### Major Criteria for Selection of Patients for Study

Have histologically or cytologically confirmed advanced (unresectable) or metastatic BTC, including intrahepatic or extrahepatic CCA, gallbladder cancer and carcinoma of Ampulla of Vater. This includes clinical diagnosis of BTC with histological confirmation of adenocarcinoma.
Have received and failed one and only one prior line of systemic treatment for advanced or metastatic disease with radiologic evidence of disease progression. This prior line of systemic treatment must also contain gemcitabine.
Have not been previously treated with varlitinib or capecitabine as first line therapy for advanced or metastatic disease. For patients who have previously received capecitabine as radiosensitizer or as part of their adjuvant therapy and their disease has relapsed for more than 6 months after their last dose of capecitabine adjuvant therapy, their capecitabine therapy will not be considered as a line of systemic chemotherapy for metastatic/advanced disease, and thus they can participate in the study.
Have radiographically measurable disease based on Response Evaluation Criteria in Solid Tumors (RECIST) v1.1.
Have no evidence of biliary duct obstruction, unless obstruction is controlled by local treatment or, in whom the biliary tree can be decompressed by endoscopic or percutaneous stenting with subsequent reduction in bilirubin to below or equal to 1.5×upper level of normal.
Have no known metastatic brain lesion(s), including asymptomatic and well controlled lesion(s).

### Statistical Analysis

The study has not been formally powered for hypothesis testing, however, it has been designed to provide a clear distinction between historical response rates of 6-8% ORR in similar populations treated with capecitabine monotherapy with a 95% confidence interval.

### Study Results

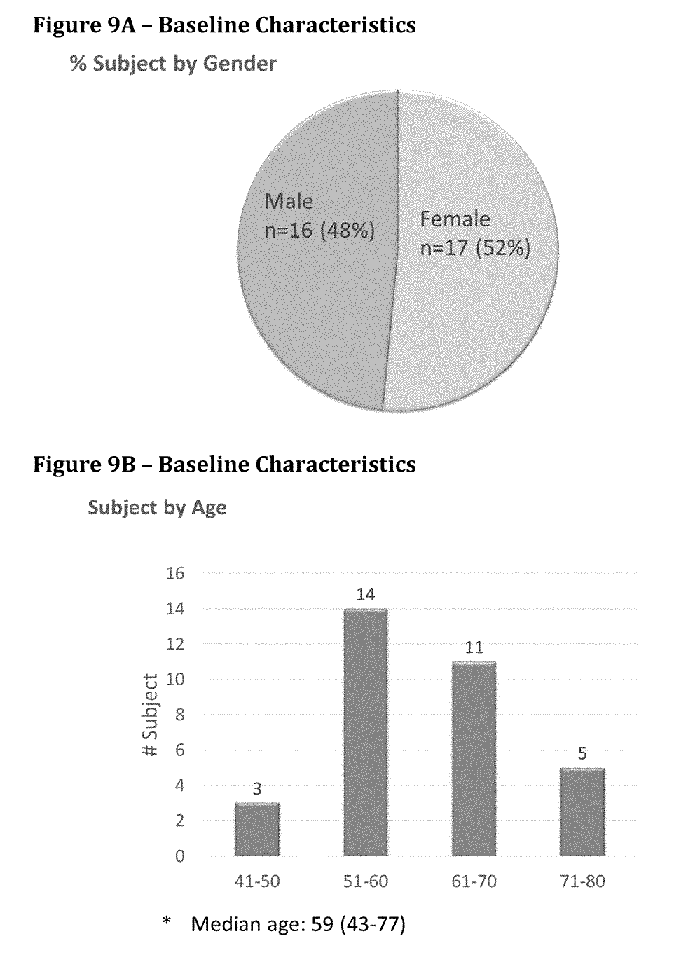

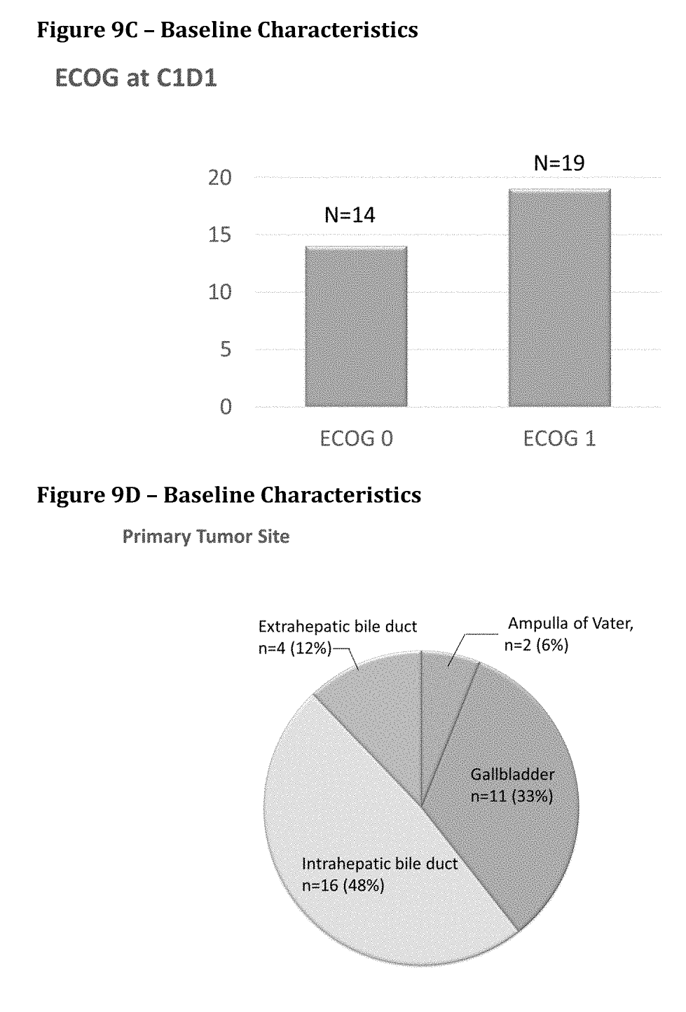

The enrollment started in December, 2017. As of 3 Sep. 2018, 46 patients have been screened and 33 patients were enrolled while 3 patients are in screening. The results of the study are shown in FIGS. 9A-9D.

### Summary

The results suggest that the combination treatment of Varlitinib and capecitabine is safe and tolerated without any unexpected adverse events. The study is ongoing and approximately 68 patients will be recruited in the study. Clinical trial identification: ClinicalTrials.gov NCT03231176

### Example 11—A Multicenter, Phase 1B Study of Varlitinib in Combination with Gemcitabine and Cisplatin (Gem/Cis) for Treatment Naïve Advanced or Metastatic Biliary Tract Cancer (BTC)

The primary objectives for the phase Ib study are to determine the maximum tolerated dose (MTD) and to characterize the safety profile of varlitinib when given with gem/cis. Secondary objectives include evaluation of preliminary efficacy of the study regimen and to evaluate the pharmacokinetics of varlitinib, and any circulating metabolites. A modified 3+3+3 escalation design was used, with 2 varlitinib dose levels (200 and 300 mg twice daily) combined with cisplatin (25 mg/m², IV for 2 hours), followed by Gemcitabine (1000 mg/m², 30-minute infusion) on Day 1 and Day 8 in a 3 weeks cycle.

### Results

### Overall Response Rate (ORR) Overview

| | | Cohort of | Cohort of |
|---|---|---|---|
| | All | 200 mg BID | 300 mg BID |
| No. of Pts Dosed | 21 | 11 | 10 |
| No. of evaluable | 21 | 11 | 10 |
| patients* | | | |
| Responders | | | |
| Complete Response | 0 | 0 | 0 |
| Partial Response | 7 | 4 | 3 |
| | (33.3%) | (36.4%) | (30%) |
| Non-responders | | | |
| SD (≥6 weeks) | 12 | 7 | 5 |
| PD | 0 | 0 | 0 |
| Inevaluable\hat{ } | 2 | 0 | 2 |

*Based on the intention-to-treat principle and includes all treated patients with measurable disease at baseline
\hat{ }Patients discontinued prior to week 6 radiological assessments.

### Disease Control Rate (DCR) Overview

| | | Cohort | Cohort |
|---|---|---|---|
| | All | of 200 mg BID | of 300 mg BID |
| No. of Pts Dosed | 21 | 11 | 10 |
| No. evaluable patients | 21 | 11 | 10 |
| Responders | 17 | 10 | 7 |
| | (81.0%) | (90.9%) | (70.0%) |
| Complete Response | 0 | 0 | 0 |
| Partial Response | 7 | 4 | 3 |
| SD (≥12 weeks) | 10 | 6 | 4 |
| Non-responders | 4 | 1 | 3 |
| SD (≤12 weeks) | 2 | 1 | 1 |
| PD | 0 | 0 | 0 |
| Inevaluable\hat{ } | 2 | 0 | 2 |

As of 10 Sep. 2018, 21 patients were enrolled (11 in 200 mg cohort and 10 in 300 mg cohort). Dose limiting toxicities were observed in 3 patients (1 G3 unconjugated hyperbilirubinemia and 1 G3 ALT Elevation/G4 AST Elevation in 200 mg cohort; 1 G4 thrombocytopenia/G3 febrile neutropenia/G3 AST elevation in 300 mg cohort). The most common (≥30%) all-grade adverse events (AEs) regardless of study drug relationship were platelet decreased (52%), neutrophil decreased (38%), decreased appetite (38%), nausea (38%), diarrhoea (38%), and anaemia (33%); the most common (≥15%) Grade ≥3 AEs were neutrophil decreased (33%), platelet decreased (29%), and anaemia (19%). Disease control rate among the 19 evaluable patients is 81.0% (7 patients had partial response per RECIST v1.1 and 10 patients achieved stable disease more than 12 weeks). At data cut-off, the median progression-free survival (PFS) for 200 mg cohort is 238 days and not reached for 300 mg cohort.

### CONCLUSIONS

Varlitinib plus gem/cis was well tolerated in the 200 mg cohort. Antitumor activity was observed for both 200 mg and 300 mg cohorts. The study is ongoing. Clinical trial information: NCT02992340.

1. A method of treating a biliary duct cancer by administering a therapeutically effective amount of a compound of formula (I): 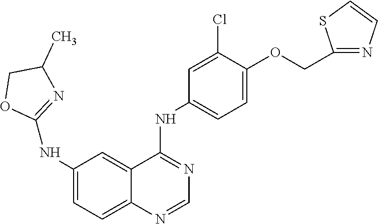 [SMILES: CC1COC(Nc2ccc3ncnc(Nc4ccc(OCc5nccs5)c(Cl)c4)c3c2)=N1]
an enantiomer thereof or a pharmaceutically acceptable salt of any one of the same.

2. A method according to claim 1, for treating a cholangiocarcinoma patient by administering a therapeutically effective amount of a compound of formula (I): 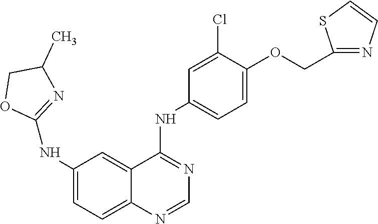 [SMILES: CC1COC(Nc2ccc3ncnc(Nc4ccc(OCc5nccs5)c(Cl)c4)c3c2)=N1]
an enantiomer thereof and/or pharmaceutically acceptable salts thereof.

3. A method according to claim 1, wherein the biliary duct cancer is gallbladder cancer.

4. A method according to claim 2, wherein the cholangiocarcinoma is located in an intrahepatic bile duct, left hepatic duct, right hepatic duct, common hepatic duct, cystic duct, common bile duct, Ampulla of Vater or a combinations thereof.

5. A method according to claim 2, wherein the cholangiocarcinoma is intrahepatic.

6. A method according to claim 2, wherein the cholangiocarcinoma is extrahepatic.

7. A method according to claim 2, wherein the compound of formula (I) is Varlitinib: 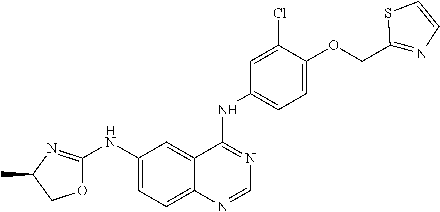 [SMILES: C[C@@H]1COC(Nc2ccc3ncnc(Nc4ccc(OCc5nccs5)c(Cl)c4)c3c2)=N1]
or a pharmaceutically acceptable salt thereof.

8. A method according to claim 2, wherein the compound of formula (I) is provided as the free base.

9. A method according to claim 2, wherein the compound of formula (I) is administered as a pharmaceutical formulation.

10. A method according to claim 2, wherein the compound of formula (I) or a pharmaceutical formulation comprising same is administered orally.

11. A method according to claim 2, wherein the compound of formula (I) or a pharmaceutical formulation comprising the same is administered bi-daily.

12. A method according to claim 2, wherein each dose of the compound of formula (I) is in the range 100 to 900 mg.

13. A method according to claim 2, wherein each dose of the compound of formula (I) is 200 mg.

14. A method according to claim 2, wherein each dose of the compound of formula (I) is 300 mg.

15. A method according to claim 2, wherein each dose of the compound of formula (I) is administered bi-daily.

16. A method according to claim 2, wherein each dose of the compound of formula (I) is in the range 300 to 500 mg.

17. A method according to claim 15, wherein each dose is 400 mg.

18. A method according to claim 2, wherein the compound of formula (I) or formulation comprising the same is employed as a monotherapy.

19. A method according to claim 2, wherein the compound of formula (I) is employed in a combination therapy.

20. A method according to claim 18, wherein the combination therapy comprises Herceptin and/or pertuzumab.

21. A method according to claim 18, wherein the combination therapy comprise ado-trastuzumab.

22. A method according to claim 18 wherein the combination therapy comprises a chemotherapeutic agent.

23. A method according to claim 21, wherein the chemotherapeutic agent is independently from the group comprising a platin (such as cisplatin or oxaliplatin), gemcitabine, capecitabine, 5-FU, FOLFOX, FOLFIRI and FOLFIRINOX.

24. A method according to claim 22, wherein the combination therapy comprises capecitabine.

25. A method according to claim 23, wherein each dose of capecitabine is 1000 mg/m².

26. A method according to claim 24, wherein each dose of capecitabine is administered bi-daily.

27. A method according to claim 22, wherein the combination therapy comprises gemcitabine and cisplatin.

28. A method according to claim 26, wherein each dose of gemcitabine is 1000 mg/m².

29. A method according to claim 26, wherein each dose of cisplatin is 25 mg/m².

30. A method according to claim 26, wherein each dose of gemcitabine and cisplatin is administered every 7 days.

31. A method according to claim 26, wherein each dose of gemcitabine and cisplatin is administered on Days 1 and 8, every 3 weeks.
